(19) United States
(12) Patent Application Publication
(10) Pub. No.: US 2014/0161848 A1
(43) Pub. Date: Jun. 12, 2014

(54) UV PROTECTIVE COSMETIC COMPOSITION

(71) Applicants: Yosuke Ikebe, Yokohama-shi (JP); Takuya Hiruma, Yokohama-shi (JP); Taichi Harada, Yokohama-shi (JP); Kazuto Fukuhara, Yokohama-shi (JP)

(72) Inventors: Yosuke Ikebe, Yokohama-shi (JP); Takuya Hiruma, Yokohama-shi (JP); Taichi Harada, Yokohama-shi (JP); Kazuto Fukuhara, Yokohama-shi (JP)

(73) Assignee: SHISEIDO COMPANY, LTD., Tokyo (JP)

(21) Appl. No.: 13/971,128

(22) Filed: Aug. 20, 2013

(30) Foreign Application Priority Data
Aug. 21, 2012 (JP) 2012-182066

(51) Int. Cl. A61K 8/36; A61K 8/368; A61K 8/29; A61K 8/27; A61Q 17/04

(52) U.S. Cl. CPC A61K 8/36; A61K 8/27; A61Q 17/04; A61K 8/29; A61K 8/368
USPC 424/401; 424/59; 424/60

(57) ABSTRACT

The present invention aims to provide a UV protective cosmetic composition having a high SPF and having a protective effect in a broad wavelength region over UVA and UVB, without undesirable white appearance upon application to the skin. The UV protective cosmetic composition of the present invention comprises (a) 3-15% by mass of one or more UV absorbing agents; (b) 10-22% by mass of zinc oxide with an average particle diameter of 35-80 nm; and optionally (c) 0-0.5% by mass of a white pigment, wherein the SPF of the cosmetic is 30 or greater, and the critical wavelength of the cosmetic is 370 nm or greater.

### CROSS REFERENCE TO RELATED APPLICATIONS

This application claims priority to JP App. Ser. No. 2012-182066 filed Aug. 21, 2012, the entire contents of which are incorporated herein fully by reference.

### FIGURE SELECTED FOR PUBLICATION

None

### BACKGROUND OF THE INVENTION

1. Field of the Invention

The present invention relates to a UV protective cosmetic composition which can protect ultraviolet rays in UVA and UVB regions in a proper balance while having a high SPF. In particular, the present invention relates to a UV protective cosmetic composition containing UV absorbing agents, zinc oxide, and optionally a white pigment in an amount equal to or less than a predetermined level.

2. Description of the Related Art

It is one of the important issues for skin care and body care to protect the skin from ultraviolet rays. It has been known that not only medium wavelength ultraviolet rays (UVB: wavelength of 290-320 nm) cause tanning, inflammation and the like, but also long wavelength ultraviolet rays (UVA: 320-400 nm) affect the skin (such as photoaging). Various cosmetics protecting the skin from these ultraviolet rays have been proposed.

Conventionally, a UV protective effect of a UV protective (sunscreen) cosmetic is predominantly expressed as SPF (Sun Protection Factor) for UVB, PFA (Protection Factor of UVA) for UVA. Currently, according to the final rule of the U.S. Food and Drug Administration (FDA) published in June 2011, the Broad Spectrum test has been adopted for a UV protective cosmetic, and a new concept of critical wavelength has been introduced in the test (Federal Register, Vol. 76, No. 117, pages 35620-35665, Jun. 17, 2011), the entire contents of which are incorporated herein by reference.

Critical wavelength (λc) is a value defined by the following formula:

$${\int }_{290}^{λ c} A ( λ ) d λ = 0.9 {\int }_{290}^{400} A ( λ ) d λ \qquad [ Numerical Expression 1 ]$$

In brief, a predetermined plate is applied with a UV protective cosmetic, irradiated with 4 MED of light, and then measured for its absorption spectrum. When an integrated value of absorbance from 290 nm to 400 nm in the above absorption spectrum is expressed as 100%, the critical wavelength (λc) is defined as the wavelength at which an absorbance value integrated from 290 nm in 1 nm steps reaches 90%. According to the final rule, only the products that have a critical wavelength (λc) of 370 nm or greater are permitted to be sold with a “broad spectrum” label.

It is believed that the above revision of the rule aims to reflect the protective effect against UVA that may be responsible for photoaging and the like in product labeling, whereas the conventional standards focused on SPF indicating the protective effect against UVB that causes sunburn.

In order to meet the requirement that the product should have a critical wavelength (λc) of 370 nm or greater, the product must have the waveform of the absorption spectrum that has the same level of absorbance in the UVA region as absorbance in the UVB region. In other words, it is important to balance the protective effect of UVB with that of UVA. In particular in the case of a high SPF formulation, the corresponding level of UV protective ability in the UVA region needs to be conferred.

In order to improve UV protective ability in the UVA region, cosmetics usually contain substances that absorb light in the UVA region as UV absorbing agents, and contain inorganic pigments such as titanium dioxide and zinc oxide as UV scattering agents. Among them, UV scattering agents, which exhibit a UV protective effect by physically scattering and absorbing ultraviolet rays on their surface, have been known to cause an undesirable unnatural whiteness when applied to the skin. To date, approaches of microparticulation (to particle diameter in the order of 15-50 nm) and controlling the shape of powders to make the white color of UV scattering agents less noticeable have been done (“Cosmetic science guide”, supervised by Tagami Hachiro et al., Fragrance Journal Ltd., published in 2007, p. 227).

JP-A 2007-161648, the entire contents of which are incorporated by reference, describes that when incorporating fine particle zinc oxide with a primary particle diameter of 15-55 nm, an average dispersed particle diameter is controlled to be 70-140 nm using a dispersing agent with a specific structure to obtain the cosmetic having an excellent UV protective effect in the UVA and UVB region, as well as providing a clear and natural finish and showing a good stability over time.

### SUMMARY OF THE INVENTION

However, fine particle zinc oxides as described in the above-mentioned publications have an insufficient UV protective effect in the UVA region (in particular, in 370-400 nm wavelength region). Therefore, in order to achieve the critical wavelength of 370 nm in a high SPF formulation, the formulation needs to be added a large amount of a white pigment such as titanium dioxide, thereby resulting in an undesirable unnatural whiteness when applied to the skin.

The present inventors made a diligent study to solve the above problem, and found that, in a UV protective cosmetic that has a high (30 or greater) SPF due to containing UV absorbing agents, incorporating zinc oxide with a larger particle size than that of conventional fine particle zinc oxide leads to a significant higher protective effect in wavelength of 370-400 nm, and leads to achievement of a critical wavelength of 370 nm or greater without necessarily adding a white pigment, and thus have accomplished the present invention.

In other words, the present invention provides a UV protective cosmetic composition comprising (a) 3-15% by mass of one or more UV absorbing agents; (b) 10-22% by mass of zinc oxide with an average particle diameter of 35-80 nm; and optionally (c) 0-0.5% by mass of a white pigment, wherein the SPF of the cosmetic is 30 or greater, and the critical wavelength of the cosmetic is 370 nm or greater.

The UV protective cosmetic composition of the present invention, which contains zinc oxide with a predetermined particle diameter and having a superior UV protective ability in wavelength of 370-400 nm, together with UV absorbing agents, has a high UV protective effect in a broad wavelength region over UVA and UVB, achieves a high SPF as high as 30 or greater and a critical wavelength of 370 nm or greater, and thus meets the final rule's requirement of U.S. FDA. Furthermore, since it contains at most 0.5% by mass of a white pigment such as titanium dioxide, an undesirable white appearance upon application to the skin does not occur.

### DETAILED DESCRIPTION OF THE PREFERRED EMBODIMENTS

The UV protective cosmetic of the present invention contains (a) one or more UV absorbing agents. The UV absorbing agents used in the present invention are selected from those which may be contained in agents for external skin application such as cosmetics, and not especially limited.

Specific examples of the UV absorbing agent may include organic UV absorbing agents such as ethylhexyl methoxycinnamate, octocrylene, polysilicone-15, butyl methoxydibenzoylmethane, ethylhexyl triazone, diethylamino hydroxybenzoyl hexyl benzoate, bis-ethylhexyloxyphenol methoxyphenyl triazine, benzophenone-3, methylene bis-benzotriazolyl tetramethylbutylphenol, phenylbenzimidazole sulfonic acid, homosalate, and ethylhexyl salicylate. Among these, combination of ethylhexyl methoxycinnamate and octocrylene is particularly preferred in view of achievement of Broad Spectrum.

The content of UV absorbing agents in the cosmetic of the present invention is such that the cosmetic can achieve an SPF of 30 or greater, and is specifically 3-15% by mass, and preferably 5-15% by mass. These numerical ranges include their lower limit and higher limit as well as all values therebetween, for example, 4, 5, 6, 7, 8, 9, 10, 11, 12, 13 and 14% by mass. The cosmetic composition containing less than 3% by mass of UV absorbing agents does not have a sufficient SPF (30 or greater), while the cosmetic composition containing over 15 Z by mass not only has difficulty in achieving a critical wavelength of 370 nm or greater, but also causes a greasy sensation on the skin when applied, tends to make the formulation unstable, and causes concern about irritation to the skin.

The UV protective cosmetic composition of the present invention contains (b) zinc oxide with an average particle diameter of 35-80 nm.

Zinc oxide used in the present invention is not especially limited as long as it has an average particle diameter of 35-80 nm, preferably 50-80 nm. It may be the zinc oxide generally used in cosmetics. The average particle diameter includes all values between the lower limit of 35 nm and the upper limit of 80 nm, for example, 40, 45, 50, 55, 60, 65, 70, 75, as well as every value or range therebetween, for example, 50-80 nm and the like. Preferably, zinc oxide with an excellent dispersibility, for example, those surface-hydrophobized by a known method as necessary may be used.

Examples of a method of surface treatment include treatment with silicone such as methylhydrogenpolysiloxane and methylpolysiloxane; treatment with fluorine such as perfluoroalkyl phosphoric acid ester and perfluoroalcohol; and treatment with amino acid such as N-acyl glutamic acid; as well as treatment with lecithin; treatment with metal soap; treatment with fatty acid; and treatment with the alkyl phosphoric acid ester. In particular, zinc oxide surface-treated with silicone is preferably used.

Silicones used in surface treatment include, but not especially limited to, various silicone oils such as methylpolysiloxane, methylphenylpolysiloxane, methylhydrogenpolysiloxane, methylcyclopolysiloxane, octamethylcyclotetrasiloxane, decamethylcyclopentasiloxane, dodecamethylcyclohexasiloxane, octamethyltrisiloxane, tetradecamethylhexasiloxane, dimethylsiloxane-methyl (polyoxyethylene) siloxane-methyl (polyoxypropylene) siloxane copolymer, dimethylsiloxane-methyl (polyoxyethylene) siloxane copolymer, dimethylsiloxane-methyl (polyoxypropylene) siloxane copolymer, dimethylsiloxane-methylcetyloxysiloxane copolymer, and dimethylsiloxane-methylstearoxysiloxane copolymer. Preferred are methylhydrogenpolysiloxane and methylpolysiloxane.

Zinc oxide used in the present invention may be commercially available, and specific examples include MZ-300 (without surface treatment agent, particle size: 35 nm, manufactured by Tayca Corporation), MZY-303S (treated with hydrogen dimethicone, particle size: 35 nm, manufactured by Tayca Corporation), MZ-306X (treated with triethoxysilylethyl polydimethylsiloxyethyl hexyl dimethicone, particle size: 35 nm, manufactured by Tayca Corporation), MZ-200 (without surface treatment agent, particle size: 50 nm, manufactured by Tayca Corporation), MZY-203S (treated with hydrogen dimethicone, particle size: 50 nm, manufactured by Tayca Corporation), MZ-150 (without surface treatment agent, particle size: 80 nm, manufactured by Tayca Corporation), MZY-153S (treated with hydrogen dimethicone, particle size: 80 nm, manufactured by Tayca Corporation), FINEX-25 (without surface treatment agent, particle size: 60 nm, manufactured by Sakai Chemical Industry Co., Ltd.), FINEX-25LP (treated with dimethicone, particle size: 60 nm, manufactured by Sakai Chemical Industry Co., Ltd.) and the like. However, it is not limited to these examples.

The cosmetic composition of the present invention is characterized in that UV protection performance in the UVA region is increased by using zinc oxide with an average particle diameter (average primary particle diameter) of 35-80 nm, which is larger than that of conventional fine particle zinc oxide.

An average primary particle diameter in the present invention means the diameter of a primary particle measured by a method generally used for zinc oxide, titanium dioxide and the like, and is specifically the particle size determined, for example, from transmission electron micrograph as arithmetic average of the longest axis and the shortest axis of a particle, or by a laser scattering/diffraction method.

The form of a zinc oxide particle is not especially limited, and the particle may be a primary particle or a secondary aggregate thereof. The shape of a particle, such as spherical, oval or fractured form, is not also especially limited.

The content of zinc oxide in the cosmetic of the present invention is 10-22% by mass, and preferably 11-20% by mass. These numerical ranges include their lower limit and higher limit as well as all values therebetween, for example, 12, 13, 14, 15, 16, 17, 18, and 19% by mass. The cosmetic composition containing less than 10 mass % of zinc oxide has a difficulty in achieving a critical wavelength of 370 nm or greater, while the cosmetic composition containing over 22 mass % tends to appear too white when applied to the skin.

The UV protective cosmetic composition of the present invention may optionally contain (c) a white pigment. The white pigment used in the present invention may be selected from white pigments usually contained in agents for external skin application such as cosmetics, and representative examples of the white pigment include titanium dioxide and zinc oxide (with the proviso that said (b) zinc oxide with an average particle diameter of 35-80 nm is excluded).

The white pigment used in the present invention may be surface-treated, or subjected to the above surface treatment as described for zinc oxide.

The particle diameter of the white pigment is preferably such that it exhibits a high light scattering effect due to Mie scattering and geometric optical scattering, for example, preferably as large as so-called pigment-grade (200-400 nm). Among white pigments, titanium dioxide with an excellent screening effect is preferably used.

Titanium dioxide used in the present invention may be commercially available, and specific examples of the commercially available titanium dioxide include Tipaque A-100 (anatase type, no surface treatment, particle size: 0.4 μm, manufactured by ISHIHARA SANGYO KAISHA, LTD.), Kronos KA-10 (anatase type, no treatment, particle size: 0.3-0.5 μm, manufactured by Titan Kogyo, Ltd.), Kronos KA-15 (anatase type, no treatment, particle size: 0.3-0.5 μm, manufactured by Titan Kogyo, Ltd.), Kronos KA-20 (anatase type, treated with aluminium oxide, particle size: 0.3-0.5 μm, manufactured by Titan Kogyo, Ltd.), Kronos KA-30 (anatase type, no treatment, particle size: 0.2-0.4 μm, manufactured by Titan Kogyo, Ltd.), Kronos KA-35 (anatase type, no treatment, particle size: 0.2-0.4 μm, manufactured by Titan Kogyo, Ltd.), Kronos KA-80 (anatase type, treated with aluminium oxide, treated with silicon dioxide, particle size: 0.3-0.5 μM, manufactured by Titan Kogyo, Ltd.), Kronos KR-310 (rutile type, no treatment, particle size: 0.3-0.5 μm, manufactured by Titan Kogyo, Ltd.), Kronos KR-380 (rutile type, treated with aluminium oxide, treated with silicon dioxide, particle size: 0.3-0.5 μm, manufactured by Titan Kogyo, Ltd.), Kronos KR-460 (rutile type, treated with aluminium oxide, particle size: 0.2-0.4 μm, manufactured by Titan Kogyo, Ltd.), Kronos KR-480 (rutile type, treated with aluminium oxide, treated with silicon dioxide, particle size: 0.2-0.4 μm, manufactured by Titan Kogyo, Ltd.), Kronos KR-270 (rutile type, treated with zinc oxide, treated with aluminium oxide, particle size: 0.2-0.4 μm, manufactured by Titan Kogyo, Ltd.), Titanix JR-301 (rutile type, treated with aluminium oxide, particle size: 0.3 μM, manufactured by Tayca Corporation), Titanix JR-403 (rutile type, treated with aluminium oxide, treated with silicon dioxide, particle size: 0.25 μm, manufactured by Tayca Corporation), Titanix JR-405 (rutile type, treated with aluminium oxide, particle size: 0.21 μm, manufactured by Tayca Corporation), Titanix JR-600A (rutile type, treated with aluminium oxide, particle size: 0.25 μm, manufactured by Tayca Corporation), Titanix JR-605 (rutile type, treated with aluminium oxide, particle size: 0.25 μm, manufactured by Tayca Corporation), Titanix JR-600E (rutile type, treated with aluminium oxide, particle size: 0.27 μm, manufactured by Tayca Corporation), Titanix JR-603 (rutile type, treated with aluminium oxide, treated with zirconium oxide, particle size: 0.28 μm, manufactured by Tayca Corporation), Titanix JR-805 (rutile type, treated with aluminium oxide and silicon dioxide, particle size: 0.29 μm, manufactured by Tayca Corporation), Titanix JR-806 (rutile type, treated with aluminium oxide, treated with silicon dioxide, particle size: 0.25 μm, manufactured by Tayca Corporation), Titanix JR-701 (rutile type, treated with aluminium oxide, treated with silicon dioxide, treated with zinc oxide, particle size: 0.27 μm, manufactured by Tayca Corporation), Titanix JRNC (rutile type, treated with aluminium oxide, treated with silicon dioxide, treated with zirconium oxide, manufactured by Tayca Corporation), Titanix JR-800 (rutile type, treated with aluminium oxide, treated with silicon dioxide, manufactured by Tayca Corporation), Titanix JR (rutile type, no treatment, particle size: 0.27 μm, manufactured by Tayca Corporation), Titanix JA-1 (anatase type, no treatment, particle size: 0.18 μm, manufactured by Tayca Corporation), Titanix JA-C (anatase type, no treatment, particle size: 0.18 μm, manufactured by Tayca Corporation), Titanix JA-3 (anatase type, no treatment, particle size: 0.18 μm, manufactured by Tayca Corporation), Titanix JA-4 (anatase type, treated with aluminium oxide, particle size: 0.18 μm, manufactured by Tayca Corporation), and Titanix JA-5 (anatase type, no treatment, particle size: 0.18 μm, manufactured by Tayca Corporation). However, it is not limited to these examples.

The lower limit of the content of a white pigment in the cosmetic of the present invention is not specifically restricted, and it may normally be about 0.001% by mass. On the other hand, the upper limit of the content of a white pigment is at most 0.5% by mass. Thus, the content of a white pigment may be 0.001-5% by mass, for example, 0.01, 0.05, 0.1, 0.2, 0.3, 0.4 or 0.5% by mass. The cosmetic composition containing over 0.5 mass % of a white pigment appears very unnaturally white when applied to the skin.

The UV protective cosmetic of the present invention is characterized in that it has an SPF of 30 or greater. SPF is a numerical value to indicate how many times the amount of ultraviolet rays exposed when a UV protective cosmetic is applied than not applied before the skin becomes slightly red. The SPF system has been adopted as an indication of a UVB prevention effect in many countries in the world. Although standards for measurement of SPF have some differences from country to country, the outlines of the measurement methods are similar (See, for example, Japan Cosmetic Industry Association SPF Measurement Standards <revised edition in 2007>, Jul. 10, 2007).

The UV protective cosmetic composition of the present invention is also characterized in that it has a critical wavelength of 370 nm or greater.

A critical wavelength is measured according to the final rule of U.S. FDA. Specifically, 0.75 mg/cm² of a UV protective cosmetic is homogeneously applied to a specified PMMA plate. Using a light source emitting continuous ultraviolet rays of 230-400 nm, the plate is irradiated with light in a four times amount (800 J/m²) of Minimal Erythema Dose (MED) of ultraviolet rays which is generally used. Then, the absorbance of the plate is measured in 1 nm steps by a spectrometer. When an integrated value of the absorbance of 290-400 nm is expressed as 100%, the wavelength at which an absorbance value integrated from 290 nm reaches 90% is defined as the critical wavelength (λc) (see Federal Register, Vol. 76, No. 117, pages 35620-35665, Jun. 17, 2011).

The UV protective cosmetic composition of the present invention achieves a critical wavelength of 370 nm or greater while having an SPF of 30 or greater by containing (a) UV absorbing agents, (b) zinc oxide with the predetermined average particle diameter, and optionally (c) titanium dioxide.

The UV protective cosmetic composition of the present invention may contain other ingredients as necessary, and the type and content of the ingredients are selected as appropriate, without impairing the advantageous effect of the present invention.

Examples of the ingredients include those usually used in cosmetics, for example, oils, moisturizing agents, surfactants, dispersing agents, water-soluble polymer, oil-soluble polymer, waxes, alcohols, hydrocarbon oils, fatty acids, higher alcohols, fatty acid esters, fragrances, preservatives, antioxidants, and drugs. However, they are not limited to these examples.

The UV protective cosmetic composition of the present invention may be provided in the appropriate form for the intended use, for example, of an emulsion, cream, gel or the like, and may be manufactured by the conventional method depending on the form of the cosmetic.

### EXAMPLES

The present invention will be described in more detail below with reference to specific examples, which is not intended to limit the technical scope of the present invention in any way. It is noted that the content in the following Examples, Comparative examples and Formulation examples are all expressed in % by mass.

The UV protective cosmetic compositions (in the form of an oil-in-water emulsion) were prepared according to the formulations shown in Table 1 and 2 below. For each cosmetic composition, an SPF and critical wavelength were measured following the above-mentioned method. Furthermore, for the degree of whiteness of the skin when each cosmetic composition is applied to the skin, 10 trained panels tested the cosmetic compositions by an actual use test. The results of the evaluation were also shown in Table 1 and 2. The degree of whiteness of the skin was rated on three scales as follows:

Evaluation scales: [symbol]
The cosmetic composition hardly appears white on the skin (It does not cause an unnatural appearance) [symbol]
The cosmetic composition appears white on the skin (It does cause a slightly unnatural appearance)
x: The cosmetic composition appears very white on the skin (It does cause an unnatural appearance)

TABLE 1

| | Exam- | Exam- | Comparative | Exam- | Exam- |
|---|---|---|---|---|---|
| Raw material name | ple 1 | ple 2 | Example 1 | ple 3 | ple 4 |
| Water | 35.05 | 34.85 | 34.65 | 31.05 | 30.95 |
| Ethanol | 6 | 6 | 6 | 6 | 6 |
| Xylitol | 1 | 1 | 1 | 1 | 1 |
| Glycerin | 2.5 | 2.5 | 2.5 | 2.5 | 2.5 |
| Dipropylene glycol | 3 | 3 | 3 | 3 | 3 |
| Distearyldimonium | 0.15 | 0.15 | 0.15 | 0.15 | 0.15 |
| hectorite | | | | | |
| PEG- | 3 | 3 | 3 | 3 | 3 |
| 10 dimethicone | | | | | |
| Isododecane | 10 | 10 | 10 | 10 | 10 |
| Dimethicone | 13.5 | 13.5 | 13.5 | 13.5 | 13.5 |
| Ethylhexyl palmitate | 3 | 3 | 3 | 3 | 3 |
| Ethylhexyl | 3 | 3 | 3 | 5 | 5 |
| methoxycinnamate | | | | | |
| Octocrylene | 3 | 3 | 3 | 3 | 3 |
| Fine particle | — | — | — | — | — |
| zinc oxide (average | | | | | |
| particle diameter: | | | | | |
| 20-30 nm)¹⁾ | | | | | |
| Zinc oxide | 11 | 11 | 11 | 13 | 13 |
| (average particle | | | | | |
| diameter: 50 nm)²⁾ | | | | | |
| White pigment³⁾ | 0.2 | 0.4 | 0.6 | 0.2 | 0.3 |
| Polymethyl- | 2.5 | 2.5 | 2.5 | 2.5 | 2.5 |
| silsesquioxane | | | | | |
| Vinyl methicone/ | 2.5 | 2.5 | 2.5 | 2.5 | 2.5 |
| methicone | | | | | |
| silsesquioxane | | | | | |
| crosspolymer | | | | | |
| EDTA-2Na | 0.2 | 0.2 | 0.2 | 0.2 | 0.2 |
| Phenoxyethanol | 0.4 | 0.4 | 0.4 | 0.4 | 0.4 |
| Total | 100 | 100 | 100 | 100 | 100 |
| Critical | 370 | 370 | 370 | 370 | 370 |
| wavelength | | | | | |
| Whiteness | ◯ | ◯ | Δ | ◯ | ◯ |
| SPF | 30> | 30> | 30> | 30> | 30> |

¹⁾Zinc oxide treated with triethoxycaprylylsilane
²⁾Zinc oxide treated with hydrogen dimethicone
³⁾Titanium dioxide treated with triethoxycaprylylsilane (particle diameter: 0.7-1.0 μm)

TABLE 2

| | | | | Comparative | Comparative | |
|---|---|---|---|---|---|---|
| Raw material name | Example 5 | Example 6 | Example 7 | example 2 | example 3 | Example 8 |
| Water | 23.25 | 24.75 | 22.75 | 22.25 | 20.75 | 19.75 |
| Ethanol | 6 | 6 | 6 | 6 | 6 | 6 |
| Xylitol | 1 | 1 | 1 | 1 | 1 | 1 |
| Glycerin | 2.5 | 2.5 | 2.5 | 2.5 | 2.5 | 2.5 |
| Dipropylene glycol | 3 | 3 | 3 | 3 | 3 | 3 |
| Distearyldimonium hectorite | 0.15 | 0.15 | 0.15 | 0.15 | 0.15 | 0.15 |
| PEG-10 dimethicone | 3 | 3 | 3 | 3 | 3 | 3 |
| Isododecane | 10 | 10 | 10 | 10 | 10 | 10 |
| Dimethicone | 13.5 | 13.5 | 13.5 | 13.5 | 13.5 | 13.5 |
| Ethylhexyl palmitate | 3 | 3 | 3 | 3 | 3 | 3 |
| Ethylhexyl methoxycinnamate | 5 | 7.5 | 7.5 | 7.5 | 7.5 | 7.5 |
| Octocrylene | 5 | 3 | 3 | 3 | 3 | 3 |
| Fine particle zinc oxide | — | — | — | 17 | 17 | — |
| (average particle diameter: | | | | | | |
| 20-30 nm)¹⁾ | | | | | | |
| Zinc oxide (average particle | — | 17 | 17 | — | — | 20 |
| diameter: 50 nm)²⁾ | | | | | | |
| Zinc oxide (average particle | 17 | — | — | — | — | — |
| diameter: 80 nm)³⁾ | | | | | | |
| Titanium dioxide treated with | 2 | — | 2 | 2 | 2 | 2 |
| stearic acid (10 nm × 50 nm) | | | | | | |
| White pigment⁴⁾ | — | — | — | 0.5 | 2 | — |
| Polymethylsilsesquioxane | 2.5 | 2.5 | 2.5 | 2.5 | 2.5 | 2.5 |
| Vinyl methicone/methicone | 2.5 | 2.5 | 2.5 | 2.5 | 2.5 | 2.5 |
| silsesquioxane crosspolymer | | | | | | |
| EDTA-2Na | 0.2 | 0.2 | 0.2 | 0.2 | 0.2 | 0.2 |
| Phenoxyethanol | 0.4 | 0.4 | 0.4 | 0.4 | 0.4 | 0.4 |
| Total | 100 | 100 | 100 | 100 | 100 | 100 |
| Critical wavelength | 372 | 371 | 371 | 367 | 371 | 371 |
| Whiteness | ∘ | ∘ | ∘ | Δ | x | ∘ |
| SPF | 30> | 30> | 30> | 30> | 30> | 30> |

¹⁾Zinc oxide treated with triethoxycaprylylsilane
²⁾Zinc oxide treated with hydrogen dimethicone
³⁾Zinc oxide treated with hydrogen dimethicone
⁴⁾Titanium dioxide treated with triethoxycaprylylsilane (particle diameter: 0.7-1.0 μm)

According to the results shown in Table 1, in the cosmetic composition that has an SPF of 30 or greater by containing 6% by mass of UV absorbing agents in total, incorporating 11% by mass of zinc oxide with an average particle diameter of 50 nm leads to achievement of a critical wavelength of 370 nm (Example 1 and 2), while Comparative example 1 containing over 0.5% by mass of a white pigment caused an unnatural whiteness. Also in Examples 3 and 4 where the UV absorbing agents were increased to 8% by mass in total to further improve an SPF, incorporating 13% by mass of zinc oxide with an average particle diameter of 50 nm and adding 0.5% by mass or less of a white raw material lead to achievement of a critical wavelength of 370 nm or greater.

With the results in Table 2, in Examples 5-8 where 17-20% by mass of zinc oxide with an average particle diameter of 50 nm or 80 nm was incorporated into the high SPF cosmetic containing 10-10.5% by mass of UV absorbing agents, a critical wavelength over 370 nm was obtained without adding a white pigment. On the other hand, in Comparative example 2 where the same amount of a conventional fine particle zinc oxide (an average particle diameter: 20-30 nm) was contained, it was found that the critical wavelength was not greater than 370 nm even when 0.5% by mass of a white pigment was added, and increasing the amount of a white pigment to 2% by mass in order to achieve a critical wavelength of 370 nm or greater caused a remarkably unnatural whiteness upon application.

1. A UV protective cosmetic composition comprising:
(a) 3 to 15% by mass of one or more UV absorbing agents; and
(b) 10 to 22% by mass of zinc oxide with an average particle diameter of 35 to 80 nm, wherein the Sun Protection Factor (SPF) of the cosmetic composition is 30 or greater, and the critical wavelength of the cosmetic composition is 370 nm or greater.

2. The cosmetic composition according to claim 1, further comprising (c) 0.001 to 0.5% by mass of a white pigment.

3. The cosmetic composition according to claim 2, wherein
the (a) UV absorbing agents are one or more selected from the group consisting of ethylhexyl methoxycinnamate, octocrylene, polysilicone-15, butyl methoxydibenzoylmethane, ethylhexyl triazone, diethylamino hydroxybenzoyl hexyl benzoate, bis-ethylhexyloxyphenol methoxyphenyl triazine, benzophenone-3, methylene bis-benzotriazolyl tetramethylbutylphenol, phenylbenzimidazole sulfonic acid, homosalate, and ethylhexyl salicylate.

4. The cosmetic composition according to claim 3, wherein the (a) UV absorbing agent consist of ethylhexyl methoxycinnamate and octocrylene.

5. The cosmetic composition according to claim 4, wherein a surface of the (b) zinc oxide is hydrophobized.

6. The cosmetic composition according to claim 5, wherein the surface of the (b) zinc oxide is treated with hydrogendimethicone.

7. The cosmetic composition according to claim 2, wherein the (c) white pigment is titanium dioxide.

8. The cosmetic composition according to claim 1, wherein the composition is an oil-in-water type emulsion.

9. A UV protective cosmetic composition comprising:
(a) 6 to 10.5% by mass of an UV absorbing agent consisting of ethylhexyl methoxycinnamate and octocrylene; and
(b) 11 to 20% by mass of zinc oxide with an average particle diameter of 35 to 80 nm, wherein the Sun Protection Factor (SPF) of the cosmetic composition is 30 or greater, and the critical wavelength of the cosmetic composition is 370 nm or greater.

10. The cosmetic composition according to claim 9, further comprising (c) 0.001 to 0.5% by mass of a titanium dioxide.

11. The cosmetic composition according to claim 1, wherein
the (a) UV absorbing agents are one or more selected from the group consisting of ethylhexyl methoxycinnamate, octocrylene, polysilicone-15, butyl methoxydibenzoylmethane, ethylhexyl triazone, diethylamino hydroxybenzoyl hexyl benzoate, bis-ethylhexyloxyphenol methoxyphenyl triazine, benzophenone-3, methylene bis-benzotriazolyl tetramethylbutylphenol, phenylbenzimidazole sulfonic acid, homosalate, and ethylhexyl salicylate.

12. The cosmetic composition according to claim 11, wherein the (a) UV absorbing agent consist of ethylhexyl methoxycinnamate and octocrylene.

13. The cosmetic composition according to claim 2, wherein the composition is an oil-in-water type emulsion.

14. The cosmetic composition according to claim 1, wherein a surface of the (b) zinc oxide is hydrophobized.

15. The cosmetic composition according to claim 14, wherein the surface of the (b) zinc oxide is treated with hydrogendimethicone.

16. The cosmetic composition according to claim 2, wherein a surface of the (b) zinc oxide is hydrophobized.

17. The cosmetic composition according to claim 16, wherein the surface of the (b) zinc oxide is treated with hydrogendimethicone.

18. The cosmetic composition according to claim 10, wherein a surface of the (b) zinc oxide is hydrophobized.

19. The cosmetic composition according to claim 18, wherein the surface of the (b) zinc oxide is treated with hydrogendimethicone.
